(19) United States
(12) Patent Application Publication
(10) Pub. No.: US 2024/0374164 A1
(43) Pub. Date: Nov. 14, 2024

(54) FUNCTIONAL THRESHOLD POWER PREDICTION USING MACHINE LEARNING

(71) Applicant: Apple Inc., Cupertino, CA (US)

(72) Inventors: Julia K. NICHOLS, Alameda, CA (US); James P. OCHS, San Francisco, CA (US); Gregory DARNELL, Palo Alto, CA (US); You REN, Seattle, WA (US); Agni KUMAR, Milton, GA (US); En-Tzu YANG, Cupertino, CA (US); Eshika MANCHANDA, Cupertino, CA (US); Britni O. CHAU, Cupertino, CA (US); Riley ROBERTS, Cupertino, CA (US)

(21) Appl. No.: 18/486,030

(22) Filed: Oct. 12, 2023

Related U.S. Application Data
(60) Provisional application No. 63/470,950, filed on Jun. 4, 2023.
(60) Provisional application No. 63/465,219, filed on May 9, 2023.

(51) Int. Cl. A61B 5/11; A61B 5/00

(52) U.S. Cl. CPC A61B 5/1118; A61B 5/7264; A61B 5/7275

(57) ABSTRACT

The subject technology provides a framework for generating physiological predictions for a user of an electronic device. The physiological predictions may include user-specific predictions of functional threshold power (FTP) that may occur if the user engages in a future activity, such as a future workout. The FTP predictions may be generated by a machine learning model that utilizes user-specific and user-agnostic physiological measures such as VO2 max to generate multiple FTP estimates according to different physiological approaches in calculating the FTP estimates and arbitrating between the FTP estimates to make the prediction with the best FTP estimate.

### CROSS-REFERENCE TO RELATED APPLICATION(S)

This application claims the benefit of U.S. Provisional Application Ser. No. 63/470,950, entitled “FUNCTIONAL THRESHOLD POWER PREDICTION USING MACHINE LEARNING” and filed on Jun. 4, 2023, and U.S. Provisional Application Ser. No. 63/465,219, entitled “FUNCTIONAL THRESHOLD POWER PREDICTION USING MACHINE LEARNING” and filed on May 9, 2023, the disclosures of which are expressly incorporated by reference herein in their entirety.

### TECHNICAL FIELD

The present description generally relates to functional threshold power prediction using machine learning applications.

### BACKGROUND

Various physiological parameters of a user can be measured and analyzed to estimate other physiological measures indicative of the user's cardiorespiratory fitness. Computer hardware has been utilized to make improvements across different industry applications including applications used to assess and monitor cardiorespiratory fitness.

### BRIEF DESCRIPTION OF THE DRAWINGS

Certain features of the subject technology are set forth in the appended claims. However, for purpose of explanation, several embodiments of the subject technology are set forth in the following figures.

FIG. 1 illustrates an example network environment in accordance with one or more implementations.

FIG. 2 illustrates an example computing architecture for a system providing for functional threshold power predictions using machine learning in accordance with one or more implementations.

FIG. 3 is a schematic diagram illustrating an example process for functional threshold power prediction in accordance with one or more implementations.

FIGS. 4A and 4B are flow charts of an example process that may be performed for generating functional threshold power predictions in accordance with one or more implementations.

FIG. 5 illustrates an example use case in which functional threshold power predictions for a user are generated responsive to a select future workout in accordance with one or more implementations.

FIG. 6 illustrates an example use case in which workout training power zones for a user are generated responsive to a select future workout in accordance with one or more implementations.

FIG. 7 illustrates an electronic system with which one or more implementations of the subject technology may be implemented.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components are shown in block diagram form in order to avoid obscuring the concepts of the subject technology.

Machine learning has seen a significant rise in popularity in recent years due to the availability of training data, and advances in more powerful and efficient computing hardware. Machine learning may utilize models that are executed to provide predictions in particular applications.

The subject technology provides techniques for providing, using machine learning, physiological predictions, such as predictions of functional threshold power (FTP) and/or other physiological information for a user of an electronic device, such that the FTP predictions can be used, for example, to generate training power zones for a future activity (e.g., high intensity activity such as cycling). The FTP predictions can be generated, from data captured by the electronic device or another electronic device of the user (e.g., training equipment such as a bike or a treadmill) and/or other users (e.g., sensor data such as heartrate sensor data, inertial measurement unit data, magnetometer data, PPG data, optical sensor data, or the like, and/or wearable workout data such as steps, speed, elevation change, and/or weather information). This data containing user activity information can be obtained and stored in a database accessible only to the electronic device of the user. These sensor signals can be used to identify best available physiological information of a user (e.g., VO2 max). In some aspects, the “best available” physiological information of a user may refer to selecting the most accurate and reliable information from the available data sources. For example, if a user is wearing a heart rate monitor, a power meter, and a GPS device during their cycling workout, the “best available” physiological information may come from the power meter, as it provides a more accurate measure of the user's workload than the heart rate monitor or GPS device. The best available VO2 max value can be used to determine multiple physiological measurement estimates of the user (e.g., FTP predictions).

Some of these FTP predictions can be determined using actual physiological measurements (e.g., heart rate deflection), actual power output measurements at certain physiological measures of the user (e.g., power at % HRmax), or by predicting physiological and/or power estimates. Other FTP predictions also can be determined by way of identifying power output estimates from historical activity information of the user. Among the multiple FTP predictions, an output FTP prediction can be obtained based on a weighted combination between the FTP predictions. The output FTP prediction can then be used to formulate training power zones for the user.

Implementations of the subject technology improve the ability of a given electronic device to provide sensor-based, machine-learning generated feedback to a user (e.g., a user of the given electronic device). These benefits therefore are understood as improving the computing functionality of a given electronic device, such as an end user device which may generally have less computational and/or power resources available than, e.g., one or more cloud-based servers.

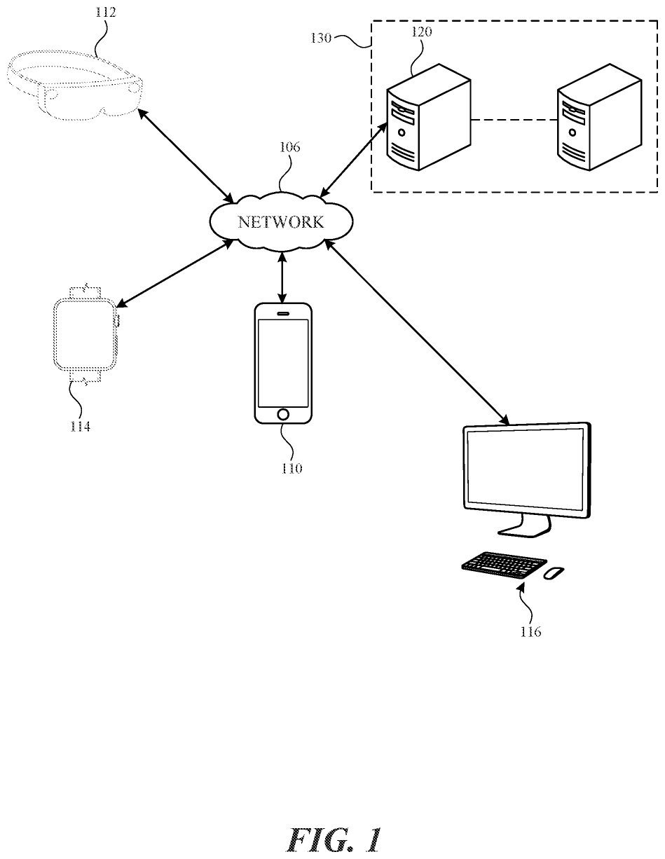

FIG. 1 illustrates an example network environment 100 in accordance with one or more implementations. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

The network environment 100 includes an electronic device 110, an electronic device 112, an electronic device 114, an electronic device 116, and a server 120. The network 106 may communicatively (directly or indirectly) couple the electronic device 110 and/or the server 120. In one or more implementations, the network 106 may be an interconnected network of devices that may include, or may be communicatively coupled to, the Internet. For explanatory purposes, the network environment 100 is illustrated in FIG. 1 as including the electronic device 110, the electronic device 112, the electronic device 114, the electronic device 116, and the server 120; however, the network environment 100 may include any number of electronic devices and any number of servers or a data center including multiple servers.

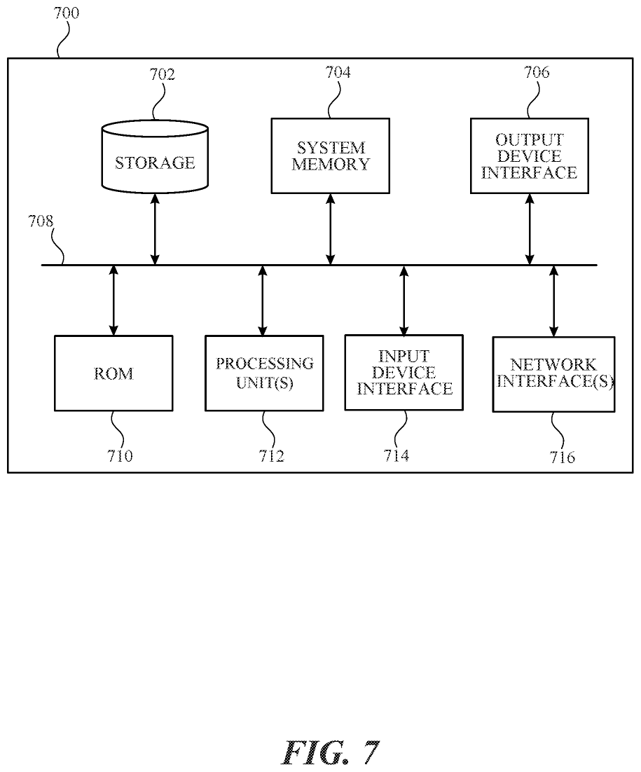

The electronic device 110 may be, for example, a desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 110 is depicted as a mobile electronic device (e.g., smartphone). The electronic device 110 may be, and/or may include all or part of, the electronic system discussed below with respect to FIG. 7.

The electronic device 112 may be, for example, desktop computer, a portable computing device such as a laptop computer, a smartphone, a peripheral device (e.g., a digital camera, headphones), a tablet device, or a wearable device such as a head mountable portable system, that includes a display system capable of presenting a visualization of an extended reality environment to a user. In FIG. 1, by way of example, the electronic device 112 is depicted as a head mountable portable system. The electronic device 112 may be, and/or may include all or part of, the electronic system discussed below with respect to FIG. 7.

The electronic device 114 may be, for example, desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 114 is depicted as a watch. The electronic device 114 may be, and/or may include all or part of, the electronic system discussed below with respect to FIG. 7.

The electronic device 116 may be, for example, desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 116 is depicted as a desktop computer. The electronic device 116 may be, and/or may include all or part of, the electronic system discussed below with respect to FIG. 7.

In one or more implementations, one or more of the electronic devices 110-116 may provide a system for training a machine learning model using training data, where the trained machine learning model is subsequently deployed to one or more of the electronic devices 110-116. Further, one or more of the electronic devices 110-116 may provide one or more machine learning frameworks for training machine learning models and/or developing applications using such machine learning models. In an example, such machine learning frameworks can provide various machine learning algorithms and models for different problem domains in machine learning. In an example, the electronic device 110 may include a deployed machine learning model that provides an output of data corresponding to a prediction or some other type of machine learning output. In one or more implementations, training and inference operations that involve individually identifiable information of a user of one or more of the electronic devices 110-116 may be performed entirely on the electronic devices 110-116, to prevent exposure of individually identifiable data to devices and/or systems that are not authorized by the user.

The server 120 may form all or part of a network of computers or a group of servers 130, such as in a cloud computing or data center implementation. For example, the server 120 stores data and software, and includes specific hardware (e.g., processors, graphics processors and other specialized or custom processors) for rendering and generating content such as graphics, images, video, audio and multi-media files. In an implementation, the server 120 may function as a cloud storage server that stores any of the aforementioned content generated by the above-discussed devices and/or the server 120.

The server 120 may provide a system for training a machine learning model using training data, where the trained machine learning model is subsequently deployed to the server 120 and/or to one or more of the electronic devices 110-116. In an implementation, the server 120 may train a given machine learning model for deployment to a client electronic device (e.g., the electronic device 110, the electronic device 112, the electronic device 114, the electronic device 116). In one or more implementations, the server 120 may train portions of the machine learning model that are trained using (e.g., anonymized) training data from a population of users, and one or more of the electronic devices 110-116 may train portions of the machine learning model that are trained using individual training data from the user of the electronic devices 110-116. The machine learning model deployed on the server 120 and/or one or more of the electronic devices 110-116 can then perform one or more machine learning algorithms. In an implementation, the server 120 provides a cloud service that utilizes the trained machine learning model and/or continually learns over time.

In the example of FIG. 1, the electronic device 110 is depicted as a smartphone. However, it is appreciated that the electronic device 110 may be implemented as another type of device, such as a wearable device (e.g., a smart watch or other wearable device) or a device mounted or integrated as part of a workout equipment (e.g., cycling machine). The electronic device 110 may be a device of a user (e.g., the electronic device 110 may be associated with and/or logged into a user account for the user at a server). Although a single electronic device 110 is shown in FIG. 1, it is appreciated that the network environment 100 may include more than one electronic device, including more than one electronic device of a user and/or one or more other electronic devices of one or more other users.

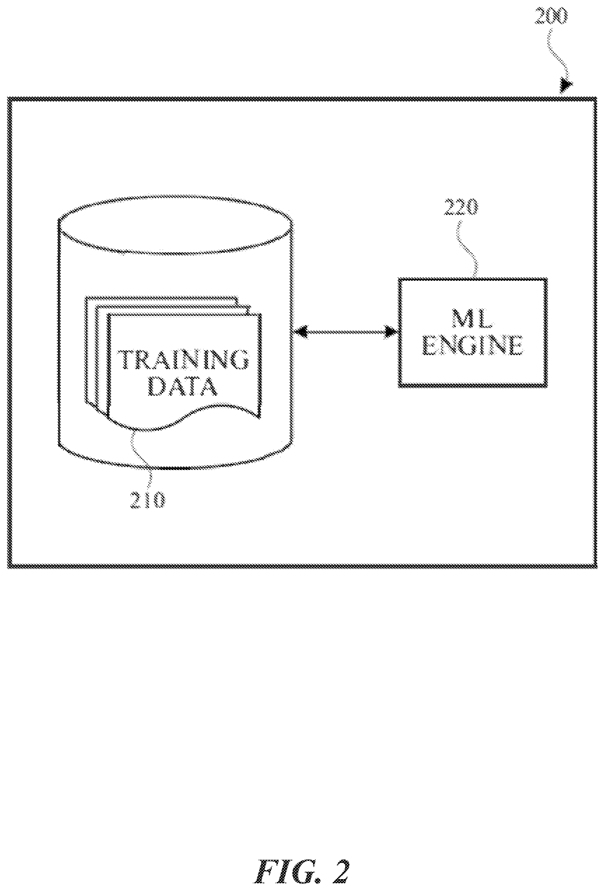

FIG. 2 illustrates an example computing architecture for a system providing machine learning models, in accordance with one or more implementations. For explanatory purposes, the computing architecture is described as being provided by an electronic device 200, such as by a processor and/or memory of the server 120, or by a processor and/or a memory of any other electronic device, such as the electronic device 110. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

As illustrated, the electronic device 200 includes training data 210 for training a machine learning model. In an example, the server 120 may utilize one or more machine learning algorithms that uses training data 210 for training a machine learning (ML) model 220.

Training data 210 may include activity information associated with activities (also referred to as events), such as workouts. For example, the activity information may include workout measurements associated with workouts and/or other activities. The workout measurements may have been obtained over the course of multiple (e.g., many) prior workouts by a user of the electronic device 110, and/or by a population of other users, such as users that were wearing wearable devices during prior workouts and/or other activities, and authorized collection of anonymized workout measurements from the wearable devices. As an example, the training data 210 may include data from, e.g., hundreds or thousands of users and/or hundreds, thousands, or millions of workouts over the course of days, weeks, months or years. In one or more implementations, training data 210 may include training data obtained by a device on which the trained ML model 220 is deployed and/or training data obtained by other devices. In one or more implementations, workout measurements included in the training data 210 may include a number of steps, a cadence, a horizontal speed (measured by a pedometer and/or a location sensor, such as a GPS sensor), an elevation change, a workout length in time or in distance, a heartrate, a blood oxygen level, and/or the like. Training data 210 may also include demographic information (e.g., age, gender, BMI, etc.) for a user of the electronic device 110, and/or a population of other users.

Training data 210 also may include workout equipment information such as power output of the workout equipment during one or more portions of a workout or other activity, the speed and/or acceleration during one or more portions of a workout or other activity, an identifier of a workout session (e.g., sessionID), an identifier of the device obtaining the workout measurements (e.g., deviceID), and/or the like. Workout measurements may also include locations (e.g., an indoor location, an outdoor location, a geographical location such as a Global Positioning System (GPS) location, or other location information) of one or more portions of a workout or other activity and/or weather conditions at the time of a workout or other activity.

For example, in one or more implementations, the training data 210 may include workout measurements contributed anonymously from more than two hundred thousand (or any number of) workouts (e.g., outdoor runs) from more than seven thousand (or any number of) subjects over a period of three years (or any number of years). The workout data may include a heartrate during each workout as well as, for example four measures of the exercise intensity: a speed from a step sensor (e.g., a pedometer), a speed from a global positioning system (GPS) sensor, a cycling cadence, and an elevation gain. The workouts from which the workout data is obtained may be, for example, between fifteen and one hundred twenty minutes long.

Machine learning model 220 may include one or more neural networks (e.g., including a latent variable model) combined with a solver for a physiological state equation, such as a VO2 max dynamics equation.

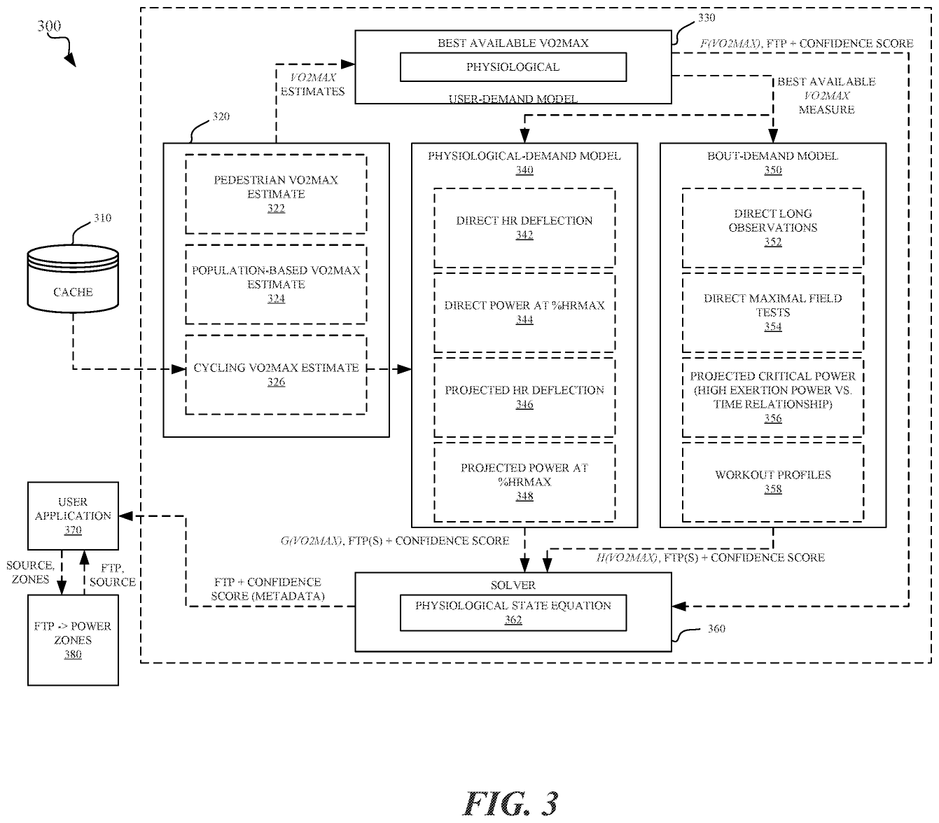

FIG. 3 is a schematic diagram illustrating an example process for functional threshold power prediction in accordance with one or more implementations. As illustrated in FIG. 3, functional threshold power predictions may be created using a metric of cardiorespiratory fitness called the VO2 max measure, which can be used to show that the learned representations disclosed herein summarize information about cardiorespiratory health. VO2 max is the maximum amount of oxygen the body can consume during exercise. This value can be measured using the heart and motion sensors on wearable devices and using demographic information such as age, gender, height, weight, and physical activity levels.

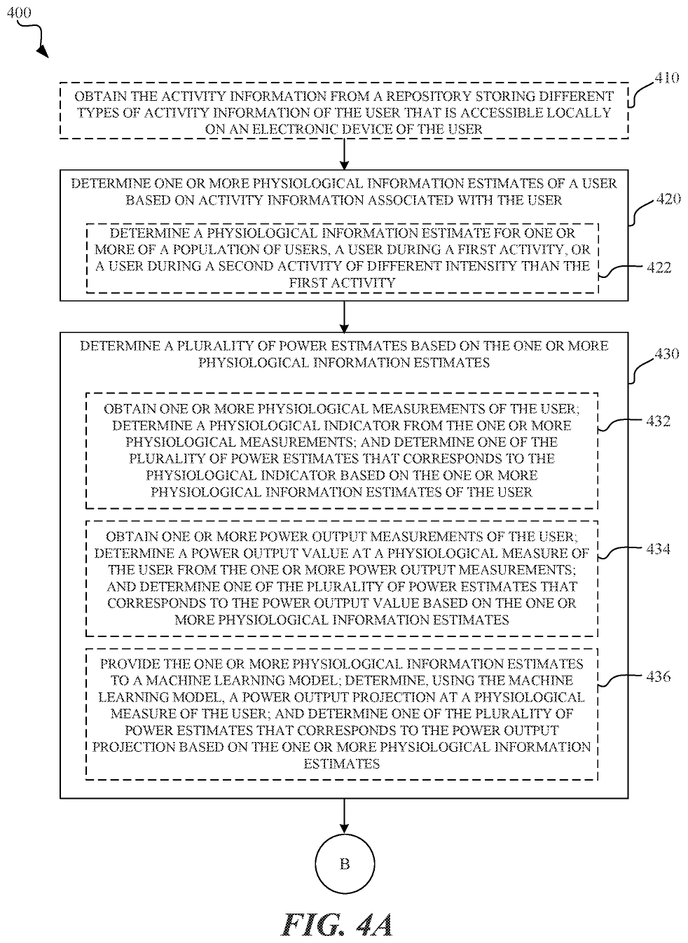

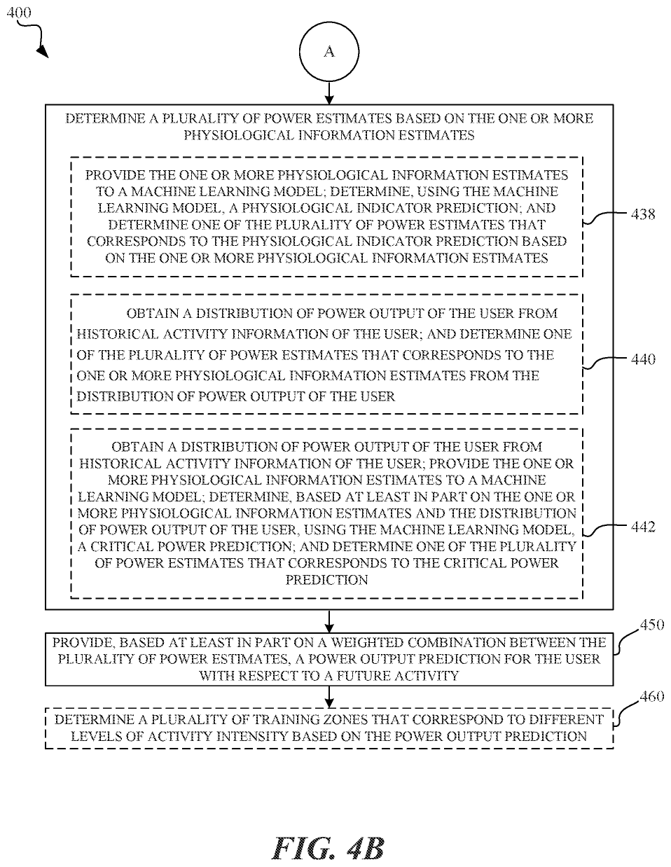

The process 300 for functional threshold power prediction will be discussed with reference to the flow charts of FIGS. 4A and 4B for ease of illustration of the subject technology. For example, FIGS. 4A and 4B are flow charts of an example process 400 that may be performed for generating functional threshold power predictions in accordance with one or more implementations. For explanatory purposes, the process 400 is primarily described herein with reference to the electronic device 110 of FIG. 1. However, the process 400 is not limited to the electronic device 110 of FIG. 1, and one or more blocks (or operations) of the process 400 may be performed by one or more other components of other suitable devices and/or servers. Further for explanatory purposes, some of the blocks of the process 400 are described herein as occurring in serial, or linearly. However, multiple blocks of the process 400 may occur in parallel. In addition, the blocks of the process 400 need not be performed in the order shown and/or one or more blocks of the process 400 need not be performed and/or can be replaced by other operations.

As illustrated in FIG. 4A, optionally, at block 410, activity information may be obtained from a repository storing different types of user activity information that is accessible locally on an electronic device of the user (e.g., the electronic device 110). As illustrated in FIG. 3, the repository may be a cache (e.g., cache 310 of FIG. 3) that is communicatively connected to the electronic device 110 for access of the user activity information on the electronic device 110. For example, the user activity information may be parameters of a workout (e.g., a cycling activity, a run, a swim, a gym activity, etc.) that has been performed by the user of a device implementing the machine learning model 220, and may include physiological measurements of a user during the workout such as a number of steps, a cadence, a horizontal speed (measured by a pedometer and/or a location sensor, such as a GPS sensor), an elevation change, a workout length in time or in distance, a heartrate, a blood oxygen level, and/or any other information that describes characteristics of the user's physiological state during the workout.

The prior workout information may be information obtained from other users that have previously performed the workout or similar activity, and/or from map data or other stored data (e.g., user-agnostic data) describing the prior workout. The activity information may also include demographic information (e.g., age, gender, BMI, etc.) for a user of the electronic device 110, and/or a population of other users. The activity information also may include workout equipment information such as power output of the workout equipment during one or more portions of a workout or other activity, the speed and/or acceleration during one or more portions of a workout or other activity, an identifier of a workout session (e.g., sessionID), an identifier of the device obtaining the workout measurements (e.g., deviceID), and/or the like.

In one or more implementations, the user activity information may include prior workout data for a population of other users, such as users that were wearing wearable devices during prior workouts and/or other activities, and authorized collection of anonymized workout measurements from the wearable devices. In other implementations, the user activity information may include prior workout data for a specific activity and/or workout (e.g., cycling, walking, or the like). Referring back to FIG. 3, a VO2 max measure model 320 can obtain the user activity information from the cache 310 to feed the user activity information into a pedestrian VO2 max estimate model 322, a population-based VO2 max estimate model 324, and a cycling VO2 max estimate model 326. In some aspects, the VO2 max measure model 320 provides a VO2 max estimate based on a population-based VO2 max estimate and/or VO2 max estimates that are user activity specific (e.g., pedestrian, cycling).

Referring back to FIG. 4A, at block 420, one or more physiological information estimates of a user may be determined based on activity information of a first activity type that is associated with the user. For example, each of the one or more physiological information estimates includes measurement information indicative of a maximum oxygen consumption of the user during an activity (e.g., VO2 max measure). For example, at block 422, a physiological information estimate may be determined for one or more of a population of users, for a user during a first activity, or for a user during a second activity of different intensity than the first activity. For example, the pedestrian VO2 max estimate model 322 can intake the user activity information pertaining to a specific individual (e.g., the user of the electronic device 110) to estimate the individual's VO2 max measure based on their walking or running activity. To estimate VO2 max for an individual, the pedestrian VO2 max estimate model 322 can analyze the user-specific physiological data from the user activity information pertaining to the walking activity, such as activity heart rate, resting heart rate, blood oxygen levels, and other metrics that can indicate cardiovascular health including various other factors such as the individual's age, gender, weight, height, and overall fitness level.

Similarly, the cycling VO2 max estimate model 326 can estimate the individual's VO2 max measure based on their cycling activity, for example, by analyzing the user-specific physiological data from the user activity information pertaining to the cycling activity. In one or more implementations, the cycling VO2 max estimate model 326 can estimate the cycling VO2 max by incorporating the pedestrian VO2 max signals.

The population-based VO2 max estimate model 324 can analyze data collected from a population rather than from an individual's actual measurements to estimate a population-based VO2 max measure. To compute a population-based VO2 max estimate, the population-based VO2 max estimate model 324 may analyze data from a representative sample of the population, including factors such as age, gender, height, weight, and physical activity levels, such that the population-based VO2 max estimate model 324 can estimate an individual's VO2 max based on their demographic and lifestyle characteristics similar to the population of users. Population-based VO2 max estimates may be useful for identifying groups of individuals who may be at higher risk for cardiovascular disease or other health conditions, and for developing targeted interventions to improve fitness and overall health.

As illustrated in FIG. 3, an implementation of the machine learning model 220 including one or more neural networks, and a solver for a physiological state equation that incorporates and/or is fed by the one or more neural networks are provided. The machine learning model 220 may include a solver 360 for a physiological state equation (PSE) 362. As shown, the user-demand model 330 may be trained to generate, based on the prior workout data, an embedding, ƒ, that is provided to the solver 360. The solver 360 may insert the embedding, ƒ, into the PSE 362, and solve the PSE 362 to generate and output one or more physiological predictions. The prior workout data from which the embedding, ƒ, is generated may be prior workout data for a user of the electronic device 110. However, in one or more use cases, prior workout data for the user of the electronic device 110 may not be available (e.g., in a case in which the electronic device 110 does not include sensors for obtaining workout data for a user, or in which no prior workout data has been previously obtained for the user of the electronic device 110). In one or more implementations, the prior workout data from which the embedding, ƒ, is generated may be prior workout data for one or more other users of one or more other electronic devices. In one or more implementations, prior workout data for one or more other users of one or more other electronic devices may be supplemented and/or selected, for generation of the embedding, ƒ, with demographic information (e.g., age, gender, BMI, etc.) for the user of the electronic device 110. In some aspects, the embedding may be a function of VO2 max (e.g., ƒ(VO2) max)).

The user-demand model 330 may be an encoder and may be implemented as a neural network (e.g., a convolutional neural network, CNN). For example, the user-demand model 330 may be a CNN with adaptive average pooling to accept variable input lengths. In one or more implementations, the embedding, ƒ, may be a learned latent representation for a user (e.g., the user of the electronic device implementing the machine learning model 220, such as the electronic device 110). Latent representations may be mathematical representations of high-dimensional data, such as physiological data, that can be learned using machine learning algorithms. For example, VO2 max is a comprehensive measure of an individual's cardiorespiratory fitness, representing their maximal capacity for oxygen consumption during exercise. As such, it can serve as a useful latent representation of a user's overall fitness level, which can be used to predict a range of physiological and performance outcomes. For example, VO2 max can be used as a learned latent representation to predict a variety of health outcomes, including cardiovascular disease, all-cause mortality, and cognitive decline. In the context of exercise and training, VO2 max can also be used to predict an individual's endurance performance and set personalized training targets. By using VO2 max as a learned latent representation of a user, it is possible to develop personalized models for predicting a wide range of physiological and performance outcomes, and to tailor training programs to individual needs and goals.

In one or more implementations, the physiological state equation 362 may be implemented as an ordinary differential equation, and may include one or more learned functions (e.g., functions having parameters that are learned by training a neural network). For example, the machine learning model 220 may include a physiological-demand model 340 and a bout-demand model 350. For example, the physiological-demand model 340 may be a function that describes the FTP of a user at a given physiological state of the user as a function of a corresponding VO2 max value. For example, the bout-demand model 350 may be a function that describes the FTP of a user for a workout bout measure at time, t, in a distribution of workout bouts as a function of a corresponding VO2 max value.

In one or more implementations, the physiological-demand model 340 and the bout-demand model 350 may each be implemented as a neural network. In this way, the parameters of functions g and h, respectively corresponding to the physiological-demand model 340 and the bout-demand model 350, can be learned by training the respective neural networks using training data, such as training data 210 of FIG. 2. For example, the physiological-demand model 340 and the bout-demand model 350 may be trained using user population training data from a population of users other than, or in addition to, the user of the electronic device 110.

In one or more implementations, the VO2 max measure model 320 can feed the VO2 max estimates from the pedestrian VO2 max estimate model 322, the population-based VO2 max estimate model 324 and the cycling VO2 max estimate model 326 as inputs to the user-demand model 330. The user-demand model 330 may be configured to determine the best available VO2 max from the input VO2 max estimates. For example, the user-demand model 330 may select the VO2 max estimate that corresponds to a desired VO2 max value. In another example, the user-demand model 330 may select the VO2 max estimate that satisfies a preconfigured VO2 max threshold.

At block 430, power estimates may be determined based on the one or more physiological information estimates. For example, once the VO2 max has been estimated for the different types of activities, they can be used to calculate the FTP of an individual, which is the maximum power output that the individual can sustain for an extended period of time. FTP is a term used in the context of cardio fitness, specifically in cycling. For example, it is defined as the highest average power output a cyclist can sustain for one hour without fatiguing excessively. FTP is an important metric for users as it helps them to gauge their current fitness level, set training goals, and monitor progress over time. Once FTP is determined, it can be used to establish training zones that correspond to different levels of intensity. These zones can be used to structure training rides and workouts to target specific physiological adaptations and improve overall fitness.

In one or more implementations, the FTP estimates may include confidence scores so that physiological predictions based on the multiple different FTP estimates can be generated to indicate an output FTP estimate for the user, and thereby recommend a custom workout with different training power zones that corresponds to the FTP prediction for which the user is predicted to sustain the maximum physiological measure or other activity level. For example, each of the power estimates includes a confidence score indicating a likelihood of a respective power estimate being a quality estimate (i.e., indicating whether the FTP estimate is indeed an accurate measure of the FTP for the user).

In one or more implementations, the FTP may be estimated using a physiological marker such as a lactate threshold, which is the exercise intensity at which blood lactate levels begin to accumulate rapidly. In some aspects, the term “lactate threshold” may also be referred to as “anaerobic threshold” as both terms may refer to the same physiological concept, and may be used interchangeably. The FTP may be referred to as the lactate threshold power, as it is often associated with the lactate threshold. The lactate threshold indicates the point at which the body begins to accumulate lactic acid in the bloodstream at a faster rate than it can be removed. This occurs during intense exercise when the body's demand for oxygen exceeds its ability to supply oxygen to the working muscles. At this point, the body begins to rely more on anaerobic metabolism, which produces lactic acid as a byproduct. The lactate threshold may be closely related to VO2 max, as it occurs at a range of VO2 max values. The lactate threshold may typically be expressed as a percentage of the VO2 max uptake. The lactate threshold can occur at around 50-80% of VO2 max, depending on individual fitness levels and the type of exercise being performed.

For example, as illustrated in FIG. 3, the user-demand model 330 may calculate the FTP as a function of VO2 max based on the corresponding lactate threshold. In this regard, the user-demand model 330, at least in part, may calculate the lactate threshold as a percentage of the best available VO2 max value determined. Based on the calculated lactate threshold, the FTP estimate can be determined. In some aspects, the FTP estimate may be accompanied by a confidence score indicating the quality measure of the FTP estimate. For example, multiple FTP estimates may be produced within a percentage range of VO2 max where the lactate threshold may occur such that each FTP estimate includes a confidence score indicating possibly a different quality measure within that percentage range of VO2 max. The FTP estimate produced by the user-demand model 330 is fed as input to the solver 360.

In one or more implementations, the physiological-demand model 340 provides a physiological approach to estimating FTP by estimating the lactate threshold. For example, optionally, at block 432, one or more physiological measurements of the user are obtained and a physiological indicator is determined from the one or more physiological measurements such that one of the power estimates that corresponds to the physiological indicator is determined based on the one or more physiological information estimates of the user. In some aspects, the physiological indicator represents a heart rate deflection data point in the one or more physiological measurements.

As discussed briefly above, the lactate threshold is closely related to FTP and can be used as a proxy for FTP estimation. To estimate the lactate threshold, the lactate threshold may be derived as a percentage of the best available VO2 max value fed from the user-demand model 330. For example, the lactate threshold may be a window in the range of 50% VO2 max to about 80% VO2 max. The heart rate deflection in the power-heart rate relationship can be detected by the direct heart rate deflection module 342. The power-heart rate relationship can refer to the relationship between power output and heart rate, which can be used to track an individual's exercise intensity. The heart rate deflection can refer to the point at which the heart rate begins to increase at a faster rate than power output, indicating that the individual has reached their lactate threshold.

The physiological-demand model 340 aims to estimate the power output at which the heart rate deflection point occurs by extrapolating from the power output curve at the lactate threshold using the known relationship between power and heart rate. For example, the direct heart rate deflection module 342 can identify the heart rate deflection point in the power-heart rate relationship until a lactate threshold point is reached within the lactate threshold window. As such, the FTP value corresponding to the lactate threshold can be determined with an accompanying confidence score. The confidence score can indicate whether the FTP estimate is indeed an accurate estimate of the FTP for the user. In some aspects, the confidence score may indicate whether the FTP estimate is a reliable measure based on its correspondence to the lactate threshold window (e.g., the confidence score can be higher for an FTP estimate corresponding to a lactate threshold inside the lactate threshold window and lower for an FTP estimate corresponding to a lactate threshold outside the lactate threshold window).

In an example, optionally, at block 434, one or more power output measurements of the user are obtained and a power output value at a physiological measure of the user is determined from the one or more power output measurements, such that one of the power estimates that corresponds to the power output value is determined based on the one or more physiological information estimates. For example, the direct power output measurements at or around a given percentage of the user's maximum heart rate can be determined by the direct power module 344. In some implementations, the heart rate in the power-heart rate relationship can be detected by the direct power module 344, such that the direct power module 344 can identify the direct power output until the user's heart rate reaches a specified heart rate (e.g., at the specified percentage of the user's maximum heart rate). For example, the direct power output can be identified in the power-heart rate relationship when the heart rate reaches 90% of the maximum heart rate. It should be appreciated that the percentage of the maximum heart rate may be a predefined parameter to the direct power module 344 or set by user configuration. In some implementations, the FTP estimate can be determined directly from the direct power output at the specified percentage of the maximum heart rate. In some aspects, the lactate threshold can be identified if the lactate threshold is reached at the specified percentage of the heart rate. As such, the FTP value corresponding to the identified lactate threshold can be determined with an accompanying confidence score. The confidence score may indicate whether the FTP estimate is indeed an accurate estimate of the FTP based on whether the lactate threshold was indeed reached.

In an example, optionally, at block 436, the one or more physiological information estimates are provided to a machine learning model, in which the machine learning model is trained to output power output predictions at different physiological measures of the user. Using the machine learning model, a power output projection at a physiological measure of the user may be determined, such that one of the power estimates that corresponds to the power output projection is determined based on the one or more physiological information estimates. For example, the direct power output can be projected for a given percentage of the user's maximum heart rate by the projected power module 348. In some implementations, the projected power module 348 can identify different power outputs for respective user heart rates including a percentage of the user's maximum heart rate according to the power-heart rate relationship. In this regard, the projected power module 348 can predict the power output when the user's heart rate reaches a percentage of the user's maximum heart rate. For example, the projected power module 348 may extrapolate power output values based on past heart rate information and project the power output value that corresponds to the heart rate reaching 90% of the maximum heart rate. As such, the FTP value corresponding to the projected power output (and/or corresponding to a physiological marker such as the lactate threshold at or near the projected power output) can be determined along with an accompanying confidence score.

Referring to FIG. 4B, in an example, optionally, at block 438, the one or more physiological information estimates are provided to a machine learning model, in which the machine learning model is trained to output physiological predictions for the user. Using the machine learning model, a physiological indicator prediction may be determined, such that one of the power estimates that corresponds to the physiological indicator prediction is determined based on the one or more physiological information estimates. For example, the physiological indicator such as the heart rate deflection point can be projected for a given power output by the projected heart rate deflection module 346. In some implementations, the projected heart rate deflection module 346 can identify different power outputs for respective user heart rates according to the power-heart rate relationship. In this regard, the projected heart rate deflection module 346 can predict the power output when the user's heart rate reaches a deflection point in the power-heart rate relationship. For example, the projected heart rate deflection module 346 may extrapolate heart rate values based on past heart rate and lactate threshold information and estimate the power output at which the projected heart rate deflection point occurs. In some implementations, the projected heart rate deflection module 346 may project the heart rate deflection point in the power-heart rate relationship until the lactate threshold point is projected to be reached. As such, the FTP value corresponding to the projected heart rate deflection point can be determined along with an accompanying confidence score.

In one or more implementations, the bout-demand model 350 provides a mechanism for estimating FTP by examining the time-power relationship during cycling bouts. To estimate FTP, the method takes into account heart rate data as well as direct long observation of user workouts. The bout-demand model 350 also uses a critical power curve to analyze cycling bouts ranging from 30 seconds to 30 minutes in duration, for example. The critical power curve projects to a critical power value, which represents the highest power output that can be sustained for a given duration without fatigue. This value can be used to estimate FTP, which represents the highest power output that can be sustained for an extended period of time. In addition to the critical power curve, the bout-demand model 350 also considers workout profiles such as interval training and high watt outputs. These profiles can provide additional information about an individual's ability to sustain high levels of power output over time. The bout-demand model 350 can perform a comprehensive analysis of cycling bouts and workout profiles to estimate FTP. By considering heart rate data, critical power curves, and workout profiles, the bout-demand model 350 can provide personalized estimates of an individual's ability to sustain high levels of power output over time. The use of machine learning algorithms and direct observation can further improve the accuracy and reliability of these estimates.

In an example, optionally, at block 440 of FIG. 4B, a distribution of power output of the user from historical activity information of the user is obtained, and one of the power estimates that corresponds to the one or more physiological information estimates is determined from the distribution of power output of the user. For example, power output values recorded over time can be analyzed by direct long observations module 352 to estimate the FTP. The average power output and VO2 max values may be recorded over an observation period of time. In some implementations, the direct long observations module 352 can calculate the FTP in accordance with the lactate threshold associated with the measured VO2 max values that correspond to the best available VO2 max value (e.g., fed by the user-demand model 330 as input to the bout-demand model 350). For example, the direct long observations module 352 can identify the FTP as the average power output that corresponds to where the lactate threshold occurs based on the corresponding VO2 max value.

In another example, average power output values corresponding to user activity at maximal effort and associated VO2 max values can be analyzed by direct maximal field test module 354 to estimate the FTP. In some implementations, the direct maximal field test module 354 may calculate the FTP as a percentage of the average power output during the observation period (e.g., using a factor of around 0.95), meaning that an individual's FTP is approximately 95% of their average power output over the measured time. In other implementations, the direct maximal field test module 354 may calculate the FTP in accordance with the lactate threshold associated with the measured VO2 max values that correspond to the best available VO2 max value (e.g., fed by the user-demand model 330 as input to the bout-demand model 350). For example, the direct maximal field test module 354 can identify the FTP as the average power output that corresponds to where the lactate threshold occurs based on the corresponding VO2 max value.

In an example, optionally, at block 442, a distribution of power output of the user from historical activity information of the user is obtained, and the one or more physiological information estimates are provided to a machine learning model, in which the machine learning model is trained to output critical power predictions for the user based at least in part on the one or more physiological information estimates and the distribution of power output of the user. Using the machine learning model, a critical power prediction is determined based at least in part on the one or more physiological information estimates and the distribution of power output of the user, such that one of the power estimates that corresponds to the critical power prediction is determined. Critical power can refer to a measure of the highest power output that an individual can maintain aerobically without fatigue for a given duration of time. The critical power can be determined through a series of high exertion activity tests (e.g., cycling tests), with the results being used by the projected critical power module 356 to construct a power-duration curve. The power-duration curve may include an extrapolation of high exertion power values indicating a projected critical power.

In some implementations, the projected critical power module 356 may identify the projected critical power point and determine the FTP directly from the projected critical power point. In other implementations, the projected critical power module 356 may identify the projected critical power point in relation to where the lactate threshold point occurs (based on the associated best available VO2 max value) such that the projected critical power module 356 determines the FTP along with a confidence score based on its correspondence to the projected critical power point. The confidence score may indicate whether the FTP estimate is indeed an accurate measure of the FTP based on a distance between the projected critical power point and the lactate threshold point with the confidence score being higher when the distance is reduced.

In some implementations, the bout-demand model 350 may determine the FTP using workout profiles containing workout bout information of one or more users. For example, workout profiles module 358 may be configured to analyze the best available VO2 max measure along with workout profile information relating to one or more users. In some implementations, the workout profiles may include a percentile of historic power. In some aspects, a first workout profile of a first user may indicate a first power curve for given VO2 max values of the first user and a second workout profile of a second user may indicate a second power curve for given VO2 max values of the second user, in which the workout profiles module 358 may determine the FTP for a subject user by identifying a weighted combination between the FTP values associated with the first and second workout profiles. The weighting may be defined by how similar one workout profile is to the workout profile of the subject user, in which the weighting increases when the similarity between the workout profiles increases.

Also referring to FIG. 4B, at block 450, a power output prediction for the user with respect to a future activity of a second activity type (e.g., cycling) different from the first activity type (e.g., walking) may be provided based at least in part on a weighted combination between the power estimates. The activity types may include walking, running, cycling, swimming, high intensity interval training, strength training, hiking, or the like. As shown in FIG. 3, the solver 360 may generate one or more physiological prediction(s) for a particular user and for a particular future activity (e.g., a particular future workout) responsive to receiving, as inputs, FTP estimates generated by the user-demand model 330 (e.g., ƒ(VO2 max), FTP+confidence score), the physiological-demand model 340 (e.g., g(VO2 max), FTP+confidence score) and the bout-demand model 350 (e.g., h(VO2 max), FTP+confidence score). As illustrated in FIG. 3, the solver 360 may provide the power output prediction to user application 370. For example, the FTP prediction along with a confidence score (and/or metadata associated with the FTP prediction) are fed to the user application for presentation to the user.

In some implementations, the FTP estimates are provided to a machine learning model as FTP estimate information prior to the user engaging in the future activity, in which the machine learning model is trained to output the power output prediction for the user based at least in part on the power estimates and corresponding confidence scores. In some aspects, the power output prediction for the user may be generated with respect to the future activity using the machine learning model and based on the FTP estimate information.

In one or more implementations, generating the power output prediction may include providing the FTP estimate information and the learned latent representation for the user to the solver 360 and generating the power output prediction with the solver 360 by solving the physiological state equation 362 using the learned latent representation for the user and the FTP estimate information. In one or more other implementations, the solver 360 may generate a deterministic solution to the physiological state equation 362. For example, in one or more implementations, the solver 360 may solve the physiological state equation 362 using an iterative operation to generate the physiological prediction(s) responsive to receiving the embedding, ƒ (representing the FTP estimate based on the best available VO2 max estimate), the FTP estimates from the physiological-demand model 340,ƒ, and/or the FTP estimates from the bout-demand model 350, g, such that the physiological state equation 362 yields a solution that is a linear weighted combination between the FTP estimates. In other implementations, the solver 360 may be implemented as a neural network trained to generate the physiological prediction(s) responsive to receiving the embedding, ƒ(VO2 max) (representing the FTP estimate based on the best available VO2 max estimate), the FTP estimates from the physiological-demand model 340, g(VO2 max), and/or the FTP estimates from the bout-demand model 350, h(VO2 max).

Aspects of the subject technology provide a hierarchical model (e.g., a hybrid machine learning model, such as the machine learning model 220) that relates the VO2 max-based parameters together. This hierarchical model can facilitate a large-scale applicability of the technology based on identifications of correlations between the VO2 max-based parameters across individuals, including their evolution over time. Because learned parameters capture the VO2 max response to exercise, they can be interpreted as summarizing the fitness level and cardio-respiratory health of various users.

As discussed herein, the bout-demand model 350 (e.g., function h (VO2 max)), the physiological-demand model 340 (e.g., function g (VO2 max)), and/or the user-demand model 330 (e.g., function ƒ(VO2 max)) can be implemented as neural networks to learn the parameters of the respective functions.

Optionally, at block 460 of FIG. 4B, training power zones that correspond to different levels of activity intensity are determined based on the power output prediction. As illustrated in FIG. 3, the user application 370 may map the FTP prediction into a set of training power zones 380 for presentation to the user. The training power zones may be defined as a percentage of the FTP value, with each zone representing a different level of effort and corresponding physiological adaptations. For example, zone 1 may represent easy endurance riding at 50-60% of FTP, while zone 5 may represent high exertion sprints at 120% or more of FTP. The specific ranges for each zone can vary depending on implementation, however, a set of power targets can be established for each training zone that are appropriate for a given individual's fitness level and training goals. In various implementations, power output predictions can be made for workouts selected by a user, and/or physiological predictions can be used to suggest a workout for a user based on physiological goals provided by a user.

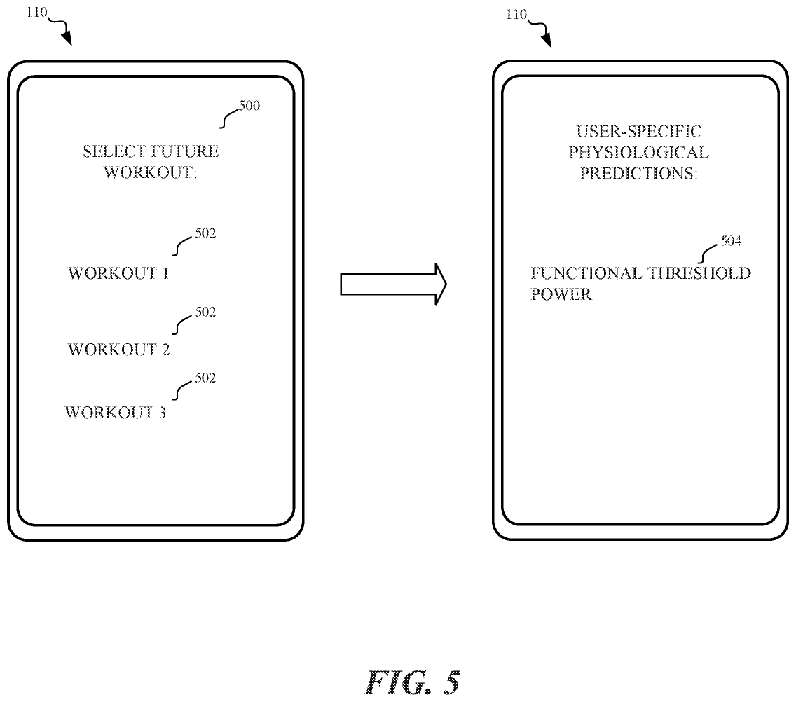

For example, FIG. 5 illustrates an example use case in which functional threshold power predictions for a user are generated responsive to a select future workout 502 in accordance with one or more implementations. For example, the future workouts 502 may be workouts that have not been previously performed by the user of the electronic device 110. The future workouts 502 may be workouts with parameters (e.g., future workout information) that are known or obtainable by the electronic device 110. For example, the future workouts 502 may correspond to runs, walks, hikes, swims, or cycles on a known route (e.g., a route having a route map that is stored at or accessible by the electronic device 110), gym activities, and/or any other workouts for which parameters (e.g., future workout information) are known or obtainable by the electronic device 110. As illustrated in FIG. 5, the user may select one of the future workouts 502 (e.g., WORKOUT 1). Responsively, the electronic device 110 may generate, using the machine learning model 220, one or more physiological predictions 504 for the selected workout for the user of the electronic device 110. As shown, the physiological predictions 504 may include a functional threshold power value that may be experienced by the user of the electronic device 110 while performing the selected workout. In various implementations, the physiological predictions 504 may include single prediction for the overall selected workout, or may include predictions as a function of time, as a function of distance during the selected workout, or as a function of the maximum oxygen consumed (e.g., VO2 max) during the selected workout. In one or more implementations, the one or more physiological predictions 504 also may include a heartrate, a heartrate range, a number of steps, a number of calories burned, a blood-oxygen level (SpO2), and/or a VO2 max.

In one illustrative use case, the user of a device implementing the machine learning model 220 may be considering cycling throughout the city. In one or more implementations, an embedding, z, for the user may have already been learned at the device of the user. Prior to the cycling activity, the user may select the cycling (e.g., by selecting an indication of a route corresponding to the cycling activity), and the device of the user may, responsively, obtain parameters of the cycling activity (e.g., cycling power, cadence, cycling speed, duration, heart rate, motion signals, user metrics including demographics, session ID, device ID, or the like). The device of the user may then provide the parameters of the cycling activity to an algorithm or the machine learning model 220 to obtain the best available VO2 max value from the cycling activity. The best available VO2 max value is then processed to extract multiple FTP estimates associated with the cycling activity. The multiple FTP estimates are then sent to the solver 360. The solver 360 may then solves the PSE 362 into which the embedding, ƒ, and multiple FTP estimates have been inserted, to generate the physiological predictions 504.

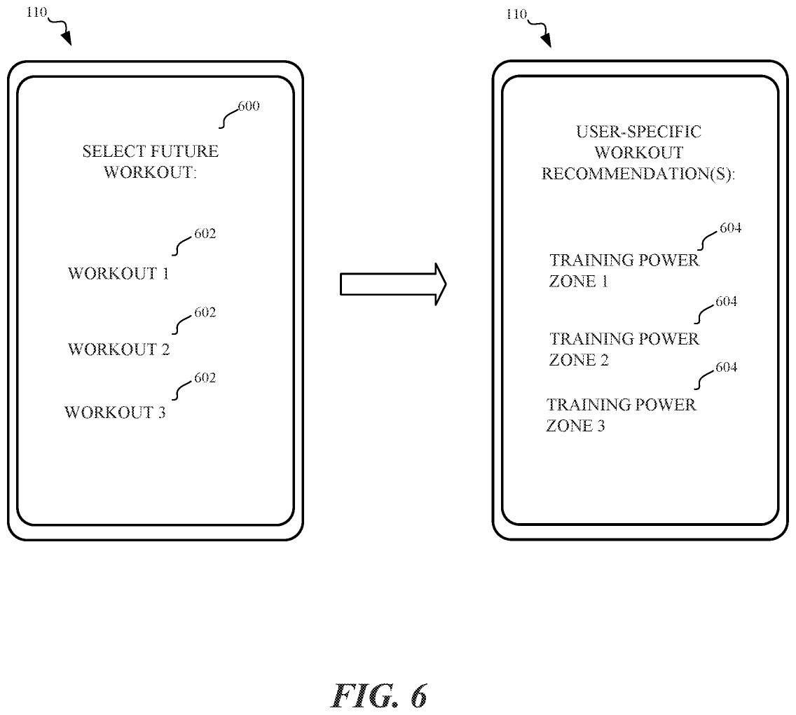

FIG. 6 illustrates an example use case in which workout training power zones for a user are generated responsive to a select future workout 602 in accordance with one or more implementations. As illustrated in FIG. 6, the user may select one of the future workouts 602 (e.g., WORKOUT 1). Responsively, the electronic device 110 may, using the machine learning model 220, generate a recommendation 604 for one (or more) of the available workouts for which physiological predictions match the user's selected future workout 602. For example, in one or more implementations, the recommendation 604 may be provided by a fitness application and may be presented in the form of a personalized workout routine that will bring the user's workout to the user's desired activity and/or physiological level(s). Signals associated with the selected future workout 602 can provide further insights into the user's training performance and can be used to generate training power zones. For example, a user may provide a request to the fitness application to provide a listing of training power zones that will cause the user to exercise for a user-selected duration of time until the user reaches its predicted physiological measure (e.g., predicted FTP value) that corresponds to one of the training power zones.

The FTP prediction (as discussed with reference to FIGS. 3-5) can be used to populate the training power zones, providing users with a personalized training plan that is tailored to their individual physiological characteristics and goals. Power zones are typically used by individuals to track and optimize training intensity, with each zone corresponding to a specific range of power output. This information may be surfaced in the workout summary, providing users with a summary of their training performance during the workout session. In some implementations, the FTP prediction may be transparent to the user. In other implementations, the FTP prediction may be provided for display to the user, as described with reference to FIG. 5.

Although physiological predictions for workouts are described herein in connection various examples, physiological predictions can be provided for activates other than workouts, such as climbing a flight of stairs, flying on an airplane, scuba diving, performing a dance, or any other physical activity.

The increased availability of wearable devices empowers individuals to track their health. The subject technology may help to quantify this measure through modelling the heart rate response to workouts. Learned representations that summarize the dynamics of the VO2 max response can serve as a measure for an individual's cardiorespiratory fitness. This measure can help track fitness level over time, provide personalized workout planning, and predict changes in cardiovascular health.

As described above, one aspect of the present technology is the gathering and use of data available from specific and legitimate sources for generating physiological predictions. The present disclosure contemplates that in some instances, this gathered data may include personal information data that uniquely identifies or can be used to identify a specific person. Such personal information data can include audio data, demographic data, location-based data, online identifiers, telephone numbers, email addresses, home addresses, biometric data or records relating to a user's health or level of fitness (e.g., vital signs measurements, medication information, exercise information, motion information, heartrate information workout information), date of birth, or any other personal information.

The present disclosure recognizes that the use of such personal information data, in the present technology, can be used to the benefit of users. For example, the personal information data can be used for generating physiological predictions.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominently and easily accessible by users, and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations which may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates aspects in which users selectively block the use of, or access to, personal information data. That is, the present disclosure contemplates that hardware and/or software elements can be provided to prevent or block access to such personal information data. For example, in the example of generating physiological predictions, the present technology can be configured to allow users to select to “opt in” or “opt out” of participation in the collection and/or sharing of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing identifiers, controlling the amount or specificity of data stored (e.g., collecting location data at city level rather than at an address level or at a scale that is insufficient for facial recognition), controlling how data is stored (e.g., aggregating data across users), and/or other methods such as differential privacy.

Therefore, although the present disclosure broadly covers use of personal information data to implement one or more various disclosed implementations, the present disclosure also contemplates that the various implementations can also be implemented without the need for accessing such personal information data. That is, the various implementations of the present technology are not rendered inoperable due to the lack of all or a portion of such personal information data.

FIG. 7 illustrates an electronic system 700 with which one or more implementations of the subject technology may be implemented. The electronic system 700 can be, and/or can be a part of, the electronic device 70, and/or the server 120 shown in FIG. 1. The electronic system 700 may include various types of computer readable media and interfaces for various other types of computer readable media. The electronic system 700 includes a bus 708, one or more processing unit(s) 712, a system memory 704 (and/or buffer), a ROM 710, a permanent storage device 702, an input device interface 714, an output device interface 706, and one or more network interfaces 716, or subsets and variations thereof.

The bus 708 collectively represents all system, peripheral, and chipset buses that communicatively connect the numerous internal devices of the electronic system 700. In one or more implementations, the bus 708 communicatively connects the one or more processing unit(s) 712 with the ROM 710, the system memory 704, and the permanent storage device 702. From these various memory units, the one or more processing unit(s) 712 retrieves instructions to execute and data to process in order to execute the processes of the subject disclosure. The one or more processing unit(s) 712 can be a single processor or a multi-core processor in different implementations.

The ROM 710 stores static data and instructions that are needed by the one or more processing unit(s) 712 and other modules of the electronic system 700. The permanent storage device 702, on the other hand, may be a read-and-write memory device. The permanent storage device 702 may be a non-volatile memory unit that stores instructions and data even when the electronic system 700 is off. In one or more implementations, a mass-storage device (such as a magnetic or optical disk and its corresponding disk drive) may be used as the permanent storage device 702.

In one or more implementations, a removable storage device (such as a floppy disk, flash drive, and its corresponding disk drive) may be used as the permanent storage device 702. Like the permanent storage device 702, the system memory 704 may be a read-and-write memory device. However, unlike the permanent storage device 702, the system memory 704 may be a volatile read-and-write memory, such as random access memory. The system memory 704 may store any of the instructions and data that one or more processing unit(s) 712 may need at runtime. In one or more implementations, the processes of the subject disclosure are stored in the system memory 704, the permanent storage device 702, and/or the ROM 710. From these various memory units, the one or more processing unit(s) 712 retrieves instructions to execute and data to process in order to execute the processes of one or more implementations.

The bus 708 also connects to the input device interface 714 and output device interface 706. The input device interface 714 enables a user to communicate information and select commands to the electronic system 700. Input devices that may be used with the input device interface 714 may include, for example, alphanumeric keyboards and pointing devices (also called “cursor control devices”). The output device interface 706 may enable, for example, the display of images generated by electronic system 700. Output devices that may be used with the output device interface 706 may include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more implementations may include devices that function as both input and output devices, such as a touchscreen. In these implementations, feedback provided to the user can be any form of sensory feedback, such as visual feedback, auditory feedback, or tactile feedback; and input from the user can be received in any form, including acoustic, speech, or tactile input.

Finally, as shown in FIG. 7, the bus 708 also couples the electronic system 700 to one or more networks and/or to one or more network nodes, such as the electronic device 110 shown in FIG. 1, through the one or more network interface(s) 716. In this manner, the electronic system 700 can be a part of a network of computers (such as a LAN, a wide area network (“WAN”), or an Intranet, or a network of networks, such as the Internet. Any or all components of the electronic system 700 can be used in conjunction with the subject disclosure.

Implementations within the scope of the present disclosure can be partially or entirely realized using a tangible computer-readable storage medium (or multiple tangible computer-readable storage media of one or more types) encoding one or more instructions. The tangible computer-readable storage medium also can be non-transitory in nature.

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device, e.g., via one or more wired connections, one or more wireless connections, or any combination thereof.

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data. Computer-executable instructions also can be organized in any format, including routines, subroutines, programs, data structures, objects, modules, applications, applets, functions, etc. As recognized by those of skill in the art, details including, but not limited to, the number, structure, sequence, and organization of instructions can vary significantly without varying the underlying logic, function, processing, and output.

While the above discussion primarily refers to microprocessor or multi-core processors that execute software, one or more implementations are performed by one or more integrated circuits, such as ASICs or FPGAs. In one or more implementations, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

As used in this specification and any claims of this application, the terms “base station”, “receiver”, “computer”, “server”, “processor”, and “memory” all refer to electronic or other technological devices. These terms exclude people or groups of people. For the purposes of the specification, the terms “display” or “displaying” means displaying on an electronic device.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The predicate words “configured to”, “operable to”, and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration”. Any embodiment described herein as “exemplary” or as an “example” is not necessarily to be construed as preferred or advantageous over other implementations. Furthermore, to the extent that the term “include”, “have”, or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for”.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

What is claimed is:

1. A method, comprising:
determining one or more physiological information estimates of a user based on activity information of a first activity type that is associated with the user;
determining a plurality of power estimates based on the one or more physiological information estimates; and
providing a power output prediction from the plurality of power estimates for the user with respect to a future activity of a second activity type different from the first activity type.

2. The method of claim 1, wherein each of the one or more physiological information estimates comprises measurement information indicative of a maximum oxygen consumption of the user during an activity.

3. The method of claim 1, further comprising obtaining the activity information from a repository storing different types of activity information of the user that is accessible locally on an electronic device of the user.

4. The method of claim 1, wherein the determining the one or more physiological information estimates comprises determining a physiological information estimate for one or more of a population of users, a user during a first activity, or a user during a second activity of different intensity than the first activity.

5. The method of claim 1, wherein the determining the plurality of power estimates comprises:
obtaining one or more physiological measurements of the user;
determining a physiological indicator from the one or more physiological measurements; and
determining one of the plurality of power estimates that corresponds to the physiological indicator based on the one or more physiological information estimates of the user.

6. The method of claim 5, wherein the physiological indicator represents a heart rate deflection data point in the one or more physiological measurements.

7. The method of claim 1, wherein the determining the plurality of power estimates comprises:
obtaining one or more power output measurements of the user;
determining a power output value at a physiological measure of the user from the one or more power output measurements; and
determining one of the plurality of power estimates that corresponds to the power output value based on the one or more physiological information estimates.

8. The method of claim 1, wherein the determining the plurality of power estimates comprises:
providing the one or more physiological information estimates to a machine learning model, wherein the machine learning model is trained to output power output predictions at different physiological measures of the user;
determining, using the machine learning model, a power output projection at a physiological measure of the user; and
determining one of the plurality of power estimates that corresponds to the power output projection based at least in part on the one or more physiological information estimates based on the one or more physiological information estimates.

9. The method of claim 1, wherein the determining the plurality of power estimates comprises:
providing the one or more physiological information estimates to a machine learning model, wherein the machine learning model is trained to output physiological predictions for the user; and
determining, using the machine learning model, a physiological indicator prediction; and
determining one of the plurality of power estimates that corresponds to the physiological indicator prediction based at least in part on the one or more physiological information estimates.

10. The method of claim 1, wherein the determining the plurality of power estimates comprises:
obtaining a distribution of power output of the user from historical activity information of the user; and
determining one of the plurality of power estimates that corresponds to the one or more physiological information estimates from the distribution of power output of the user.

11. The method of claim 1, wherein the determining the plurality of power estimates comprises:
obtaining a distribution of power output of the user from historical activity information of the user;
providing the one or more physiological information estimates to a machine learning model, wherein the machine learning model is trained to output critical power predictions for the user based at least in part on the one or more physiological information estimates and the distribution of power output of the user; and
determining, based at least in part on the one or more physiological information estimates and the distribution of power output of the user, using the machine learning model, a critical power prediction; and
determining one of the plurality of power estimates that corresponds to the critical power prediction.

12. The method of claim 1, wherein each of the plurality of power estimates includes a confidence score indicating a likelihood of a respective power estimate being a quality estimate.

13. The method of claim 1, further comprising determining a plurality of training power zones that correspond to different levels of activity intensity based on the power output prediction.

14. The method of claim 1, further comprising providing the plurality of power estimates to a machine learning model prior to the user engaging in the future activity, wherein the machine learning model is trained to output the power output prediction for the user based at least in part on a weighted combination between the plurality of power estimates and corresponding confidence scores.

15. A device, comprising:
a memory; and
one or more processors configured to:
determine one or more physiological information estimates of a user based on activity information associated with the user;
determine a plurality of power estimates based on the one or more physiological information estimates; and
provide a power output prediction for the user with respect to a future activity from the plurality of power estimates.

16. The device of claim 15, wherein the one or more processors configured to determine the plurality of power estimates are further configured to:
obtain one or more physiological measurements of the user;
determine a physiological indicator from the one or more physiological measurements; and
determine one of the plurality of power estimates that corresponds to the physiological indicator based on the one or more physiological information estimates of the user.

17. The device of claim 15, wherein the one or more processors configured to determine the plurality of power estimates are further configured to:
obtain one or more power output measurements of the user;
determine a power output value at a physiological measure of the user from the one or more power output measurements; and
determine one of the plurality of power estimates that corresponds to the power output value based on the one or more physiological information estimates.

18. The device of claim 15, wherein the one or more processors configured to determine the plurality of power estimates are further configured to:
provide the one or more physiological information estimates to a machine learning model, wherein the machine learning model is trained to output power output predictions at different physiological measures of the user;
determine, using the machine learning model, a power output projection at a physiological measure of the user; and
determining one of the plurality of power estimates that corresponds to the power output projection based on the one or more physiological information estimates.

19. The device of claim 15, wherein the one or more processors configured to determine the plurality of power estimates are further configured to:
provide the one or more physiological information estimates to a machine learning model, wherein the machine learning model is trained to output physiological predictions for the user; and
determine, using the machine learning model, a physiological indicator prediction; and
determine one of the plurality of power estimates that corresponds to the physiological indicator prediction based on the one or more physiological information estimates.

20. A non-transitory machine-readable medium comprising code that, when executed by a processor, causes the processor to perform operations comprising:
determining one or more physiological information estimates of a user based on activity information associated with the user;
determining a plurality of power estimates based on the one or more physiological information estimates; and
providing a power output prediction for the user with respect to a future activity from the plurality of power estimates.
